(19) United States
(12) Patent Application Publication
(10) Pub. No.: US 2004/0111006 A1
(43) Pub. Date: Jun. 10, 2004

(54) System and method for regulating blood pressure

(75) Inventors: Clifton A. Alferness, Redmond, WA (US); John M. Adams, Sammamish, WA (US)

Correspondence Address: Richard O. Gray, Jr., GRAYBEAL JACKSON HALEY LLP, 155 - 108th Avenue NE, Suite 350, Bellevue, WA, 98004-5901, US

(73) Assignee: Scout Medical Technologies, LLC

(21) Appl. No.: 10/322,563

(22) Filed: Dec. 17, 2002

(51) Int. Cl. A61N 1/362

(52) U.S. Cl. 600/16

(57) ABSTRACT

A blood pressure control system regulates blood pressure of a patient. The system includes a pressure sensor that senses blood pressure of a patient, a processor that determines if the blood pressure sensed by the pressure sensor is above a target pressure, and a blood flow regulator that reduces venous return blood flow in response to the processor determining that the sensed blood pressure is above the target pattern. The system may alternatively be employed for acutely reducing blood pressure in response to detected congestive heart failure episodes.

### FIELD OF THE INVENTION

The present invention generally relates to a system and method for regulating blood pressure of a patient. The present invention is more particularly directed to such a system and method wherein venous return blood flow is regulated in response to sensed blood pressure of the patient.

### BACKGROUND OF THE INVENTION

Cardiovascular diseases are the major cause of death and morbidity in the United States. These diseases result in a huge financial burden on the economy. The misery and disability that can result from cardiovascular disease is even more devastating.

High blood pressure, otherwise known as hypertension, is a condition that occurs when the pressure inside of large arteries is too high. It is a silent disorder. The only way to detect hypertension is to measure a patient's blood pressure. Hypertension is a very common problem that affects about 50 million people in the United States alone. It is more common as people grow older.

Hypertension can create significant health risks. Such risks include stroke, arteriosclerosis, heart attack, kidney damage, and enlarged hearts. With respect to stroke, high blood pressure can harm the arteries, causing the arteries to narrow faster. As a result, less blood can get to the brain. If a blood clot blocks one of the narrowed arteries, a thrombotic stroke may occur. If very high pressure causes a break in a weakened blood vessel in the brain, a hemorrhagic stroke may occur. With respect to arteriosclerosis, high blood pressure can make arteries thick and stiff. This speeds the build up of cholesterol and fats in the blood vessels which can impede the blood from flowing through the body, and in time, can lead to a heart attack or stroke. With respect to heart attack, blood carries oxygen to the body. When the arteries that bring blood to the heart muscle become blocked, the heart cannot get enough oxygen. Reduced blood flow can cause chest pain (angina). Eventually, the flow may be stopped completely, causing a heart attack. With respect to enlarged hearts, high blood pressure causes the heart to work harder. Over time, this causes the heart to thicken and/or stretch. Eventually, the heart fails to function normally causing fluids to back up into the lungs. Lastly, with respect to kidney damage, the kidneys act as a filter to rid the body of waste and control fluid volume load. Over a number of years, high blood pressure can narrow and thicken the blood vessels of the kidney. The kidney will filter less fluid, and waste builds up in the blood. The kidneys may even fail altogether. When this occurs, dialysis or a kidney transplant may be required.

As can be seen from the foregoing, high blood pressure or hypertension may result in any one of a number of debilitating conditions. Hence, there is a need in the art, for a system and method which is capable of controlling blood pressure to maintain the blood pressure within safe limits. The present invention addresses this need.

### SUMMARY OF THE INVENTION

The present invention provides a blood pressure control system. The blood pressure control system includes a pressure sensor that senses blood pressure of a patient, and a blood flow regulator that varies venous return blood flow in response to the sensed blood pressure. The system may further include a processor that determines if the blood pressure is above a target pressure and the regulator may reduce venous return blood flow if the processor determines that the sensed blood pressure is above the target pressure.

Preferably, the pressure sensor senses the blood pressure at spaced apart times. The processor preferably computes running blood pressure averages and determines if computed running blood pressure averages are above the target pressure.

The blood flow regulator may, in addition, increase the venous return blood flow if the blood pressure is below the target pressure. The blood flow regulator may controllably restrict blood flow through the inferior vena cava at a point above or below (proximal or distal to) the renal veins for controlling the venous return blood flow. The blood flow regulator may more particularly include an inflatable cuff configured to be placed about the inferior vena cava. Alternatively, the blood flow regulator may include an inflatable balloon configured to be placed within the inferior vena cava.

The system is preferably totally implantable within a human body. However, portions of the system may be external to the body.

Preferably, the processor decreases the target pressure over time. The processor may incrementally decrease the target pressure every twenty-four to seventy-two hours.

The system may further include an activity sensor that senses patient activity. The processor may then determine the target pressure responsive to the sensed patient activity.

The processor may determine a mean arterial pressure responsive to the pressure sensor and at the spaced apart times, compute average mean arterial pressures, and determines if computed average mean arterial pressures are above the target pressure. The processor may average a last predetermined number of determined mean arterial pressures to compute the average mean arterial pressures.

The pressure sensor may sense blood pressure of the left atrium to adapt the system for treating episodes such as congestive heart failure episodes. Here, the target pressure may be a fixed target pressure programmable by the physician.

The present invention still further provides a blood pressure control system comprising blood pressure sensing means for sensing blood pressure of a patient, and blood flow regulating means for varying venous return blood flow responsive to the sensed blood pressure.

The present invention still further provides a method of controlling blood pressure of a patient. The method includes the steps of sensing blood pressure of the patient, and varying venous return blood flow responsive to the sensed blood pressure.

The invention still further provides a blood pressure monitor comprising a pressure sensor adapted to be mounted on a septal membrane of a heart, and a method of monitoring blood pressure including the steps of attaching a pressure sensor to a septal membrane of a heart to generate a signal representing sensed blood pressure, and reading the signal representing the sensed blood pressure.

### BRIEF DESCRIPTION OF THE DRAWINGS

The features of the present invention which are believed to be novel are set forth with particularity in the appended claims. The invention, together with further aspects and advantages thereof, may best be understood by making reference to the following description taken in conjunction with the accompanying drawings, in the several figures of which like reference numerals identify identical elements, and wherein:

FIG. 1 is a simplified diagram of a human heart and a blood pressure control system embodying the present invention;

FIG. 2 is a block diagram of a blood pressure control system embodying the present invention;

FIG. 3 is a partial cross sectional side view of a blood pressure regulator embodying the present invention disposed about an inferior vena cava to regulate venous return blood flow;

FIG. 4 is a simplified diagram, similar to FIG. 1, of an alternative embodiment of a blood pressure control system in accordance with the present invention;

FIG. 5 is a partial side view, to an enlarged scale, illustrating details of the pressure sensor of the system of FIG. 4;

FIG. 6 is a flow diagram illustrating an overview of the operation of a blood pressure control system embodying the present invention;

FIG. 7 is a flow diagram illustrating a safety protocol which may be implemented in a blood pressure control system embodying the present invention;

FIG. 8 is a flow diagram illustrating the manner in which the target blood pressure may be determined in accordance with the present invention;

FIG. 9 is a flow diagram illustrating the manner in which the target blood pressure may be determined while taking patient activity into account in accordance with the present invention;

FIG. 10 is a simplified diagram of a blood pressure control system embodying the present invention configured for treating congestive heart failure episodes of a human heart in accordance with the present invention;

FIG. 11 is a simplified diagram of another blood pressure control system embodying the present invention configured for treating congestive heart failure episodes of a human heart in accordance with the present invention;

FIG. 12 is a flow diagram illustrating an overview of the operation of the blood pressure control systems of FIGS. 10 and 11 for treating congestive heart failure episodes in accordance with the present invention

FIG. 13 is a partial side view of another venous blood return flow regulator embodying the present invention shown disposed in an inferior vena cava; and

FIG. 14 is a partial sectional view of the blood flow regulator of FIG. 13.

### DETAILED DESCRIPTION OF THE INVENTION

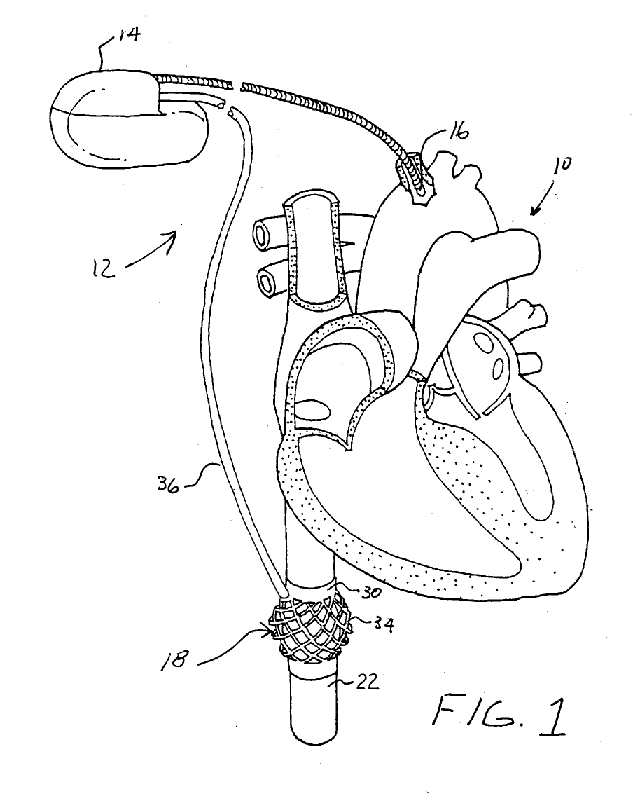

Referring now to FIG. 1, it shows a blood pressure regulating system 12 embodying the present invention in operative association with a heart 10 in need of blood pressure regulation. The system 10 generally includes a blood pressure control device 14, a blood pressure sensor 16, and a blood pressure regulator 18. The blood pressure sensor 16 is shown disposed in a carotid artery of the heart 10 but may be placed in any other available major artery, such as the descending aorta. The blood pressure regulator 18 is shown disposed on the inferior vena cava 22 (IVC) of the heart 10. As will be seen hereinafter, the device 14 is responsive to the blood pressure sensed by the sensor 16 and controls the regulator 18 to reduce venous return blood flow to the heart 10 when the blood pressure is above a target blood pressure.

The device 14 in response to the sensor 16 provides at spaced apart times a mean arterial pressure (MAP). The device includes a processor which calculates a running average of a last predetermined number of mean arterial pressures. It then compares the running averaged mean arterial pressure to a target pressure. If the running average mean arterial blood pressure is above a target pressure, the device 14 causes the regulator 18 to increase constriction of the IVC to reduce the venous return blood flow to the heart. With the return blood flow thus reduced, the heart 10 will exhibit lower cardiac output which then will in turn cause the blood pressure to drop accordingly.

As will be seen hereinafter, the target pressure is reduced gradually over long periods of time to permit the patient's body to adapt to the lower pressure as an acceptable new lower normal blood pressure. The slow reductions in cardiac output maintain peripheral vascular resistance either constant or slowly reducing. Sudden decreases in cardiac output could cause an increase in vascular resistance. As the blood pressure decreases, the body organs will accommodate this reduction by reducing their vascular resistance. Over time, a new normal blood pressure will be achieved. This new normal lower blood pressure will be achieved without causing reductions in blood flow to the brain or heart of the patient.

As a result, the blood pressure regulating system 12 overcomes the adaptation of the human body to a high blood pressure as being “normal”. The natural feedback systems in the human body to maintain a high “normal” blood pressure results in chronic hypertension. However, with controlled reduction in venous return blood flow to the heart in accordance with the present invention over an extended period of time, such as, by making each reduction every 24 to 48 hours to accommodate the baroreceptor adaptation time, the patient's body will adapt to a lower blood pressure as an acceptable new and lower “normal” blood pressure, thus relieving the hypertension.

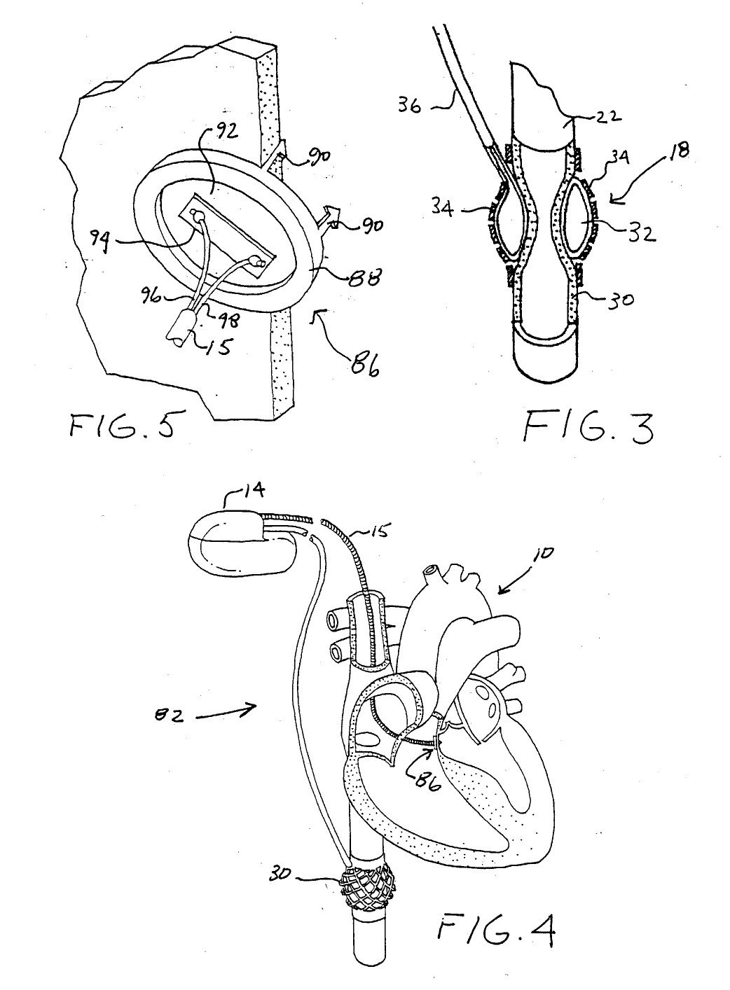

As shown in FIG. 3, the blood flow regulator 18 is a cuff 30 that encircles the IVC 22. The cuff has a fluid reservoir 32. As fluid is pumped into the fluid reservoir 32, the fluid acts upon relatively rigid or non-stretchable outer casing 34 so that the volume increase of the reservoir 32 is deflected inwardly to cause the cuff 30 to constrict the IVC and thus reduce venous return blood flow to the heart. The fluid provided to the reservoir 32 is conducted through a conduit 36 from the device 14. The relatively rigid or non-stretchable outer casing 34 may be formed of a non-stretchable woven fabric material such as, for example, fabric made from polyester, nylon, or polypropylene.

Other placements of the flow regulator 18 on the IVC are also possible. For example,-the cuff 30 may be placed about the IVC at a point below or distal to the renal veins. This has the additional advantage of assisting in averting an adverse effect or reaction of the kidney to the lower blood pressure.

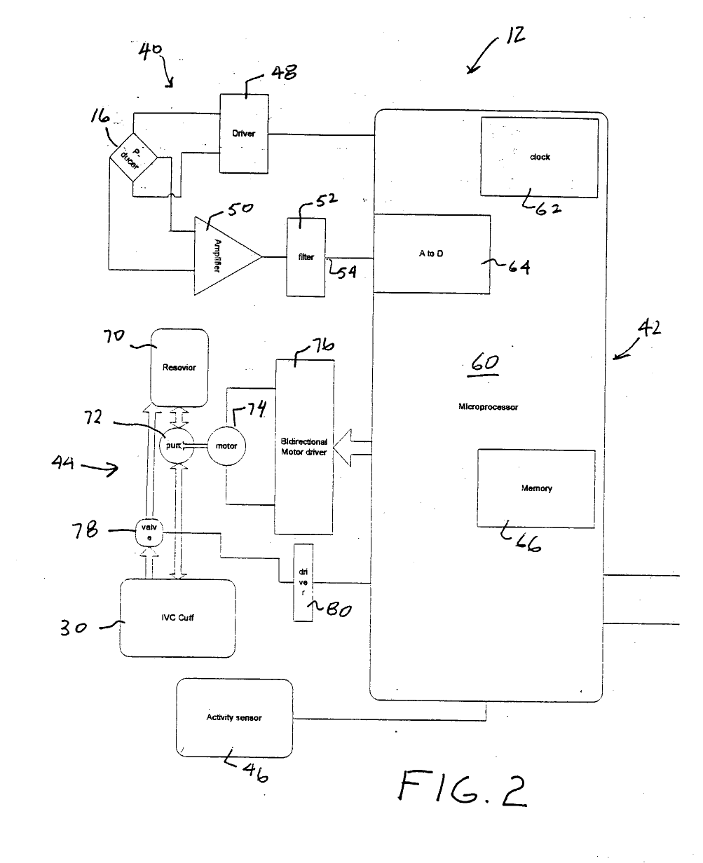

Referring now to FIG. 2, it is a block diagram of the blood pressure regulating system 12. The system generally includes a blood pressure sensing stage 40, a blood pressure control stage 42, a blood pressure regulating stage 44, and an activity sensor 46.

The blood pressure sensing stage 40 includes the blood pressure sensor 16 which comprises a pressure transducer. The blood pressure sensing stage further includes a driver 48, an amplifier 50, and a filter 52.

The control stage 42 comprises a microprocessor 60 which has a clock 62, an analog-to-digital converter 64, and a memory 66. The clock 62 keeps track of various time periods including the time between blood pressure measurements. The analog-to-digital converter converts the analog signal provided at the output 54 of the filter 52 representing the instantaneous blood pressure sensed by the pressure transducer 16 and digitizes the analog signal to a digital format for processing by the microprocessor 60. As previously mentioned, at spaced apart times, the pressure sensing stage 40 senses the blood pressure of the patient and from the digital data provided by the analog-to-digital converter 64 during the blood pressure sensing, determines a mean arterial pressure. Mean arterial pressure is well known in the art and the manner in which it is determined is also well known in the art.

The memory 66 stores data and operating instructions used by the microprocessor 60. The operating instructions stored in the memory 66 define the implementation of the microprocessor and includes the generation of a running average of a last predetermined number of mean arterial pressures. The operating instructions stored in memory 66 also implements the comparing of the running mean arterial pressure averages to a target pressure to determine if the running mean arterial pressure averages are less than or greater than the target pressure as will be described subsequently. The memory 66 also stores a look-up table which associates target blood pressures with corresponding instances in time.

The blood pressure regulating stage 44 includes the IVC cuff 30, a reservoir 70 containing the fluid which is provided to the IVC cuff 30 when further constriction of the IVC is required and which receives fluid from the IVC cuff 30 when the constriction of the IVC is decreased. To that end, the blood pressure regulating stage 44 includes a pump 72 for pumping fluid from the reservoir 70 to the IVC cuff 30 or for pumping fluid from the IVC cuff 30 back to the reservoir 70. The pump is controlled by a motor 74 which is in turn controlled by a bidirectional motor drive 76 which is in turn coupled to the microprocessor 60 from which it receives control signals defining the pumping of fluid from the reservoir into the IVC cuff or pumping of fluid from the IVC cuff to the reservoir. Lastly, the regulating stage 44 includes a valve 78 which is provided to provide quick release of fluid from the IVC cuff 30 into the reservoir 70. The valve 78 is controlled by a driver 80 which is coupled to the microprocessor 60.

As will be seen hereinafter, the target blood pressure may be modified by the activity of the patient. To that end, the system 12 includes the activity sensor 46. The activity sensor 46 may be, for example, a transducer or an accelerometer of the type well known in the art.

Referring now to FIG. 4, it illustrates another blood pressure regulating system 82 embodying the present invention for controlling the blood pressure of the heart 10. The system 82 is essentially identical to the system 12 of FIG. 1 in that it includes the device 14 and the venous return blood flow regulating cuff 30 as previously described. Here however, the system 82 includes a pressure sensor 86 which, instead of being within a carotid artery, is placed within the right ventricle on the membranous septum of the heart 10 between the right ventricle and the left ventricle. Alternatively, the pressure sensor may be placed on the membranous septum within the right atrium between the right atrium and the left ventricle. Alternatively, if left atrial blood pressure is to be monitored, the sensor may be placed on the interatrial septum between the right atrium and the left ventricle. The sensor 86 may take the form of a pressure transducer as previously described. By being placed on the septum, the pressure sensor 86 is capable of sensing the blood pressure within the left ventricle.

FIG. 5 shows details of the sensor 86. As will be noted in FIG. 5, the sensor 86 includes a rigid ring 88 which may be formed of stainless steel. Extending from the rigid ring 88 are a plurality of barbed projections 90 arranged to pierce the membranous septum for mounting the pressure sensor 86 on the membranous septum. Within the ring 88 is disposed a flexible diaphragm 92 which may be formed of silicon rubber, for example. Secured to the silicon diaphragm 92 is the transducer sensor 94 which senses the pressure in the left ventricle. Attached to the transducer 94 are a pair of conductors 96 and 98 which extend through the lead 15 to the device 14 as illustrated in FIG. 4. Preferably, the ring 88, the diaphragm 92, and the transducer 94 are encapsulated.

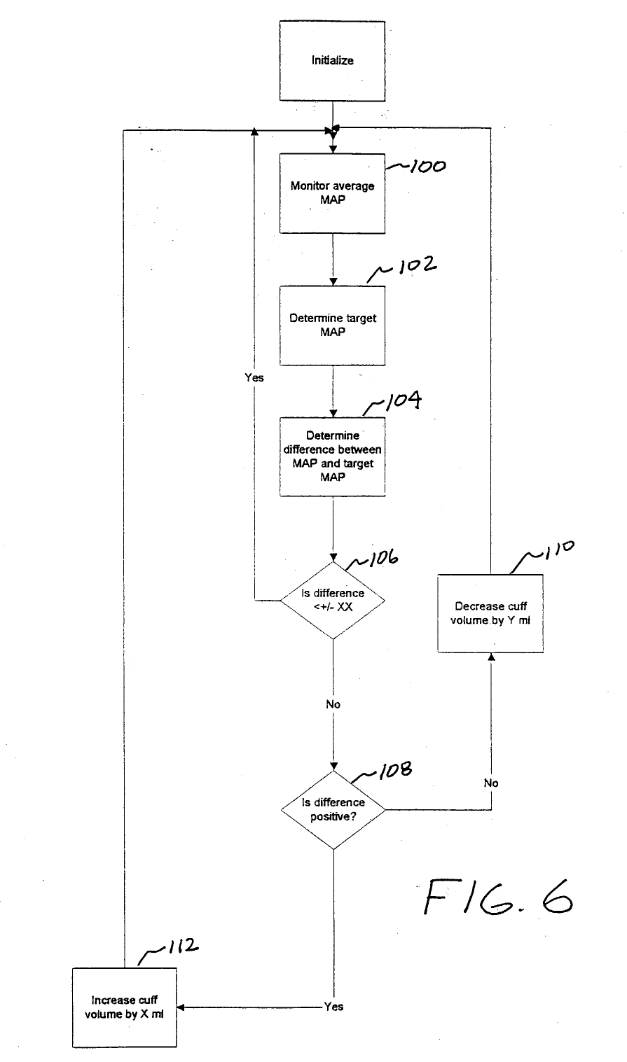

In FIG. 6, a flow diagram is shown describing an overview of the operation which may be implemented in the system of FIG. 2 in accordance with one embodiment of the present invention. In this flow diagram, and the other flow diagrams described herein, the various algorithmic steps are summarized in individual “blocks”. Such blocks describe specific actions or decisions that must be made or carried out as the algorithms proceed. Where a microcontroller (or equivalent) is employed, the flow diagrams presented herein provide the basis for a “control program” that may be used by such a microcontroller (or equivalent) to effectuate the desired operation of the system. Those skilled in the art may readily write such a control program based on the flow diagrams and other descriptions presented herein.

The operation of FIG. 6 initiates at an activity block 100 wherein an average mean arterial pressure is determined. As previously mentioned, the average determined in activity block 100 is a running average of a last predetermined number of acute mean arterial pressures. Hence, activity block 100 is contemplated to represent that at spaced apart times, an acute mean arterial pressure is determined. After the predetermined number of mean arterial pressures are determined or after a predetermined time, the acute mean arterial pressures are averaged to determine the average mean arterial pressure. As will also be seen subsequently with respect to FIG. 7, any determined mean arterial pressure may be utilized, if sufficiently below a safe limit, to cause the valve 78 (FIG. 2) to release the venous return blood flow constriction as a safety measure.

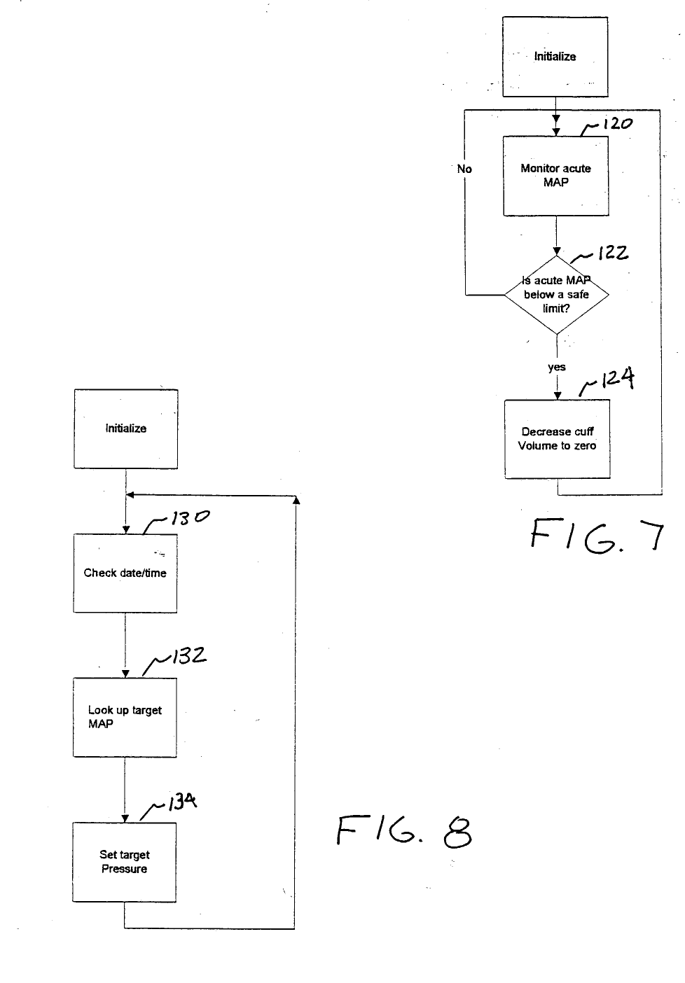

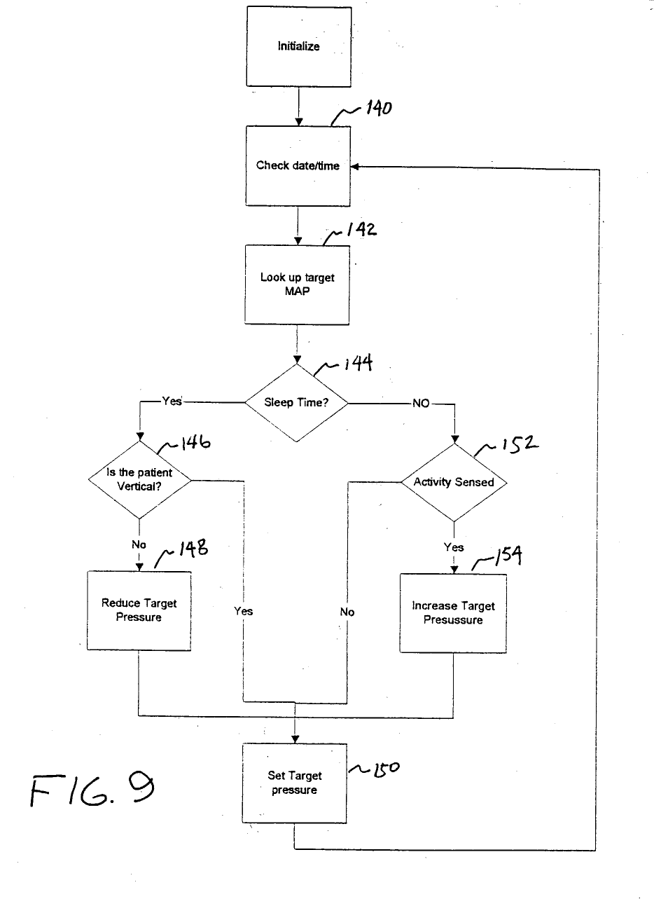

Once an average mean arterial pressure is determined, the process then advances to activity block 102 wherein the target mean arterial pressure is determined. The mean arterial pressure target may be determined, as for example, as shown in the flow diagrams of FIGS. 8 and 9 to be described hereinafter.

Once the target mean arterial pressure is determined, the process advances to activity block 104 wherein the difference between the average mean arterial pressure and the target mean arterial pressure is determined. In an implementing activity block 104, the target mean arterial pressure is subtracted from the average mean arterial pressure determined in activity block 102. Once the pressure difference is determined, the process advances to decision block 106.

In decision block 106, the microprocessor 60 determines if the pressure difference is within a range of plus or minus XX wherein XX may be 12 mm hg, for example. If the pressure difference is within this range, the process returns to activity block 100. However, if the pressure difference is outside of this range, the process then advances to decision block 108.

In decision block 108, it is determined if the pressure difference is positive. If the pressure difference is not positive, indicating that the mean arterial pressure is less than the target pressure, the process advances to activity block 110 wherein the venous return blood flow is increased. As illustrated in FIG. 6, activity block 110 may be implemented by drawing fluid out of the IVC cuff 30 into the reservoir 70 to decrease the cuff volume and thus decrease the constriction on the venous return blood flow. However, if the pressure difference is positive, indicating that the average mean arterial pressure is above the target mean arterial pressure, the process advances to activity block 112 wherein the venous return blood flow is further decreased. As further noted in FIG. 6, activity block 112 may be implemented by pumping fluid from the reservoir 70 into the IVC cuff 30 to increase the cuff volume. This will cause further constriction on the IVC and decrease the venous return blood flow. In implementing either activity block 110 or activity block 112, the decrease or increase to the cuff volume is incremental. Hence, in accordance with activity block 110, the cuff volume is decreased by an increment of Y milliliters, which may be one milliliter, for example, and in activity block 112, the cuff volume is increased by an increment of X milliliters, which may be 0.2 milliliters. Once the cuff volume has been incrementally increased or decreased as determined in decision block 108, the process returns to activity block 100.

Referring now to FIG. 7, it illustrates the previously mentioned safety protocol. This subroutine initiates at an activity block 120 wherein an acute mean arterial pressure is determined. As previously mentioned, in addition for use in the safety protocol, the determined acute mean arterial pressure is utilized for determining the next running average mean arterial pressure.

Once the acute mean arterial pressure is determined, the subroutine advances to decision block 122 wherein the microprocessor 60 determines if the determined acute mean arterial pressure is below a safe limit. If the determined mean arterial pressure is not below a safe limit, the process returns to activity block 120. However, if the determined acute mean arterial pressure is below a safe limit, the process immediately advances to activity block 124 wherein all of the constriction on the IVC is removed. This permits the maximum possible venous return blood flow to the heart. In implementing activity block 124, the microprocessor 60 causes the driver 80 to open the valve 78 to decrease the IVC cuff pressure to zero.

The flow diagram of FIG. 8 describes a manner in which the target mean arterial pressure may be determined in accordance with one embodiment of the present invention. This subroutine of activity block 102 of FIG. 6 initiates at activity block 130. In activity block 130, the microprocessor 60 determines the current date and time utilizing the clock 62. Once the current date and time are determined in activity block 130, the subroutine advances to activity block 132 wherein the microprocessor utilizes a lookup table to look up the target mean arterial pressure which corresponds to the determined day and time of activity block 130. Once the target mean arterial pressure is found in the lookup table, the process then advances to activity block 134 wherein the determined target mean arterial pressure is set by the microprocessor for the implementation of activity block 104 of FIG. 6 wherein the mean arterial pressure difference is determined.

Referring now to FIG. 9, it is a flow diagram describing an alternative method of determining the target mean arterial pressure for implementing activity block 102 of FIG. 6. Here, the microprocessor not only utilizes the date and time of day to determine the target mean arterial pressure, but in addition, the activity state of the patient.

The subroutine of FIG. 9 initiates with activity block 140. Here again, the microprocessor 60 determines from the clock 62 the date and time of day. Once the date and time of day are determined, the subroutine advances to activity block 142 wherein the microprocessor looks up in a lookup table, the target mean arterial pressure corresponding to the determined date and time. Following activity block 142, the subroutine advances to decision block 144 wherein it is determined if the time of day is such that the patient would normally be asleep. If the patient would normally be asleep, the process then advances to decision block 146 wherein it is determined, from the activity sensor, if the patient is vertically disposed. If the patient is not vertically disposed and thus at rest or asleep, the process then advances to activity block 148 wherein the target pressure determined in activity block 142 is reduced. Once the target pressure is reduced, the process then advances to activity block 150 wherein the newly determined target mean arterial pressure is set for use in activity block 104 of FIG. 6. The process then returns to activity block 140. If in decision block 146 it is determined that the patient is vertical, and thus not at rest or asleep, the process immediately advances to activity block 150 to set the target pressure at the target mean arterial pressure determined in step 142 without a reduction.

If in decision block 144 it is determined that the current time is not when the patient would normally be asleep, the process advances to decision block 152 wherein the microprocessor 60 determines from the activity sensor 46 if the patient is currently active. If the patient is not currently active, the process advances to activity block 150 wherein the target pressure is set to the mean arterial pressure determined in activity block 142. However, if in decision block 152 it is determined that the patient is currently active, the subroutine then advances to activity block 154 wherein the target pressure is increased. The process then advances to activity block 150 wherein the new increased target mean arterial pressure is set by the microprocessor for use in activity block 104 of FIG. 6.

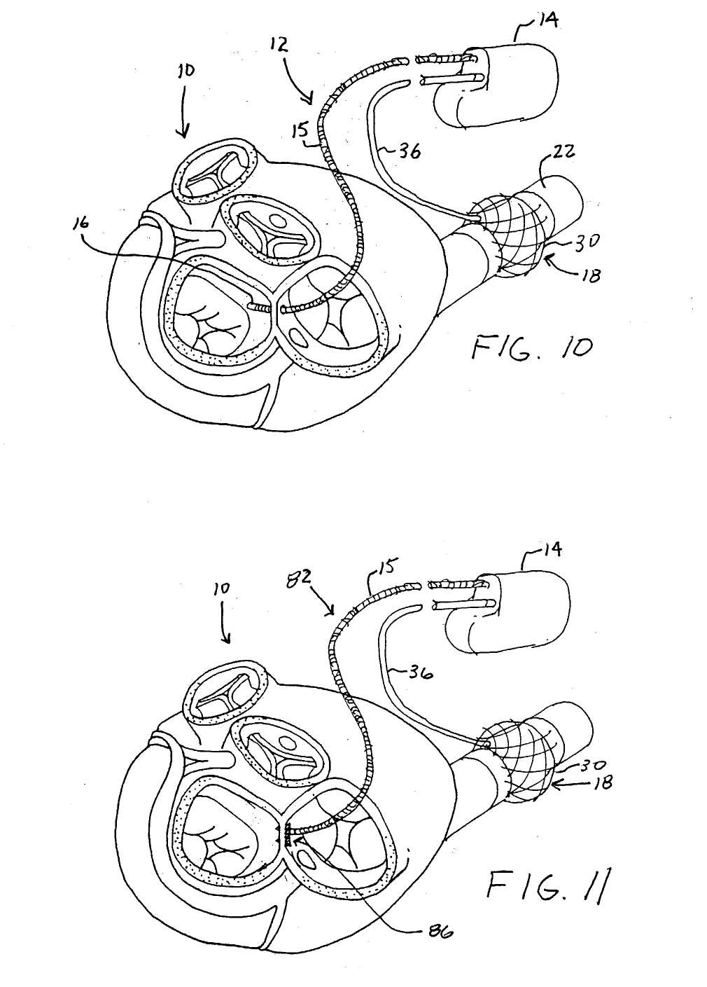

Referring now to FIG. 10, it shows the system 12 of FIG. 1 being utilized for treating high blood pressures associated with congestive heart failure attacks. In FIG. 10 it will be noted that the lead 15 of the system penetrates the interatrial septum to place the sensor 16 within the left atrium. This enables the sensor 16 to sense left atrial pressure within the heart 10.

When the left atrial blood pressure exceeds a target left atrial blood pressure, the device 14 provides additional fluid to the venous return flow regulator 18 which includes the cuff 30 through the conduit 36. This causes the cuff 30 to further restrict the venous return blood flow in the inferior vena cava 22 to decrease the patient's blood pressure. The decrease in venous return blood flow, in accordance with this embodiment, will have a higher incremental value in view of the need to acutely decrease the patient's blood pressure.

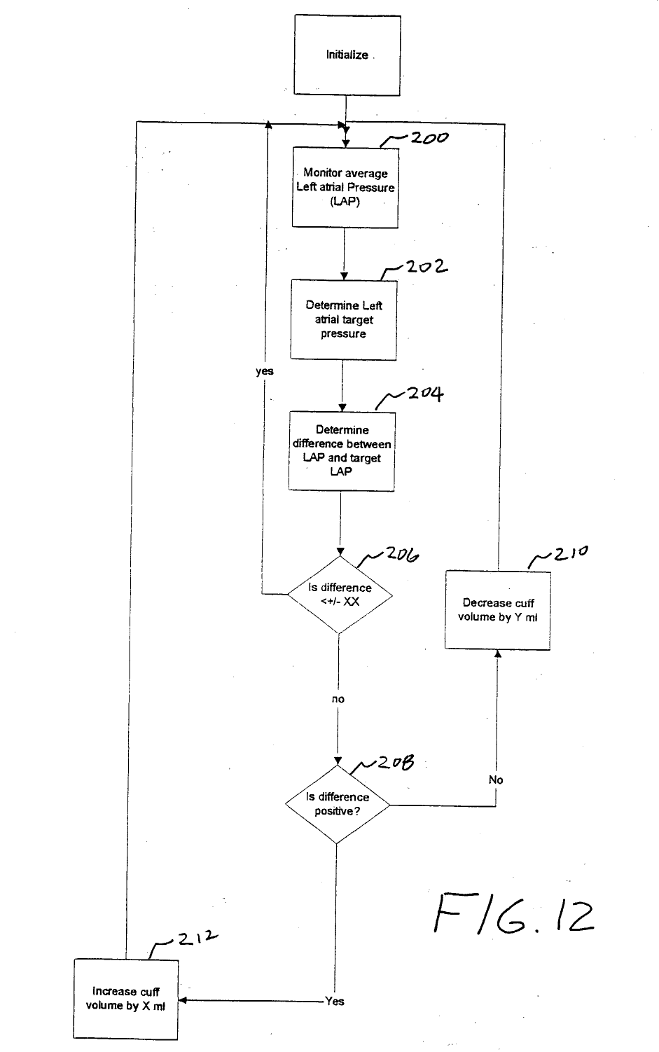

FIG. 11 illustrates the system 82 also being utilized to treat high blood pressure associated with congestive heart failure episodes. Here it will be noted that the lead 15 terminates with the pressure sensor 86, previously described, placed on the interatrial septum to sense blood pressure within the left atrium. The system 82 also includes the venous return blood flow regulator 18 including the cuff 30 and fluid conduit 36. The operation of the system 82 is identical to the operation of the system 12 as previously described.

FIG. 12 is a flow diagram describing the operation of the systems 12 and 82 as shown in FIGS. 10 and 11 respectively. In accordance with this embodiment of the present invention, the implementation of the therapy for high blood pressure associated with congestive heart failure episodes is essentially identical to the implementation described with reference to FIG. 6 for general hypertension treatment. To that end, the process illustrated in FIG. 12 initiates with an activity block 200 wherein an average left atrial pressure is determined. The average pressure may be a running average of a last predetermined number of determined left atrial blood pressures. The left atrial pressures may be mean pressures.

Once the average left atrial pressure is determined in accordance with activity block 200, the process advances to activity block 202 wherein the left atrial target blood pressure is determined. In accordance with this embodiment of the present invention, the left atrial target pressure may be a fixed target pressure set or programmed by the physician. Hence, in implementing activity block 202, the microprocessor interrogates its memory to determine the target pressure set by the physician. However, as previously described, the system does have the ability to vary the target pressure if desired.

Once the left atrial target pressure is determined in accordance with activity block 202, the process advances to activity block 204 wherein the difference between the average left atrial blood pressure determined in activity block 200 and the target left atrial blood pressure determined in 202 is determined. In accordance with this embodiment, the target left atrial blood pressure is subtracted from the averaged left atrial blood pressure.

The process then advances to decision block 206 where it is determined if the pressure difference is within a given range, plus or minus XX. If the pressure difference is within the range, the process returns. However, if the pressure is outside of that range, the process advances to decision block 208 wherein it is determined if the difference is positive. If the difference is not positive, indicating that the left atrial blood pressure is less than the target left atrial blood pressure, the process immediately advances to activity block 210 wherein the venous return blood flow is permitted to increase by decreasing the cuff volume as previously described. However, if the pressure difference is positive, the process advances to activity block 212 wherein the venous return blood flow is decreased by increasing the cuff volume as previously described in order to more acutely reduce the blood pressure of the patient suffering from a congestive heart failure episode.

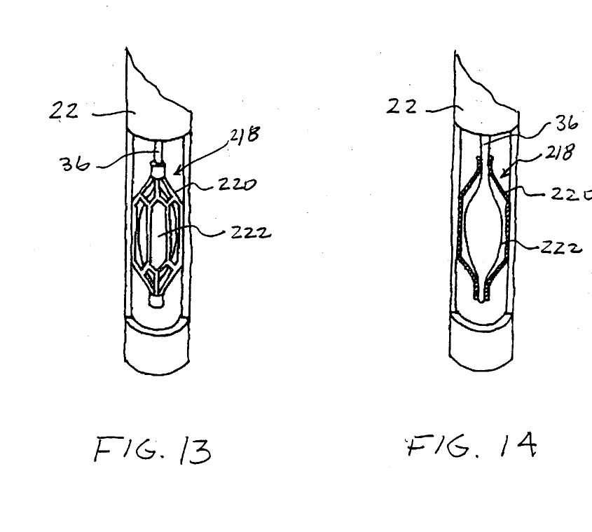

Referring now to FIGS. 13 and 14, they illustrate a further blood flow regulator 218 embodying the present invention. The blood flow regulator 218 is shown disposed within an inferior vena cava 22 for regulating venous return blood flow. The blood flow regulator 218 generally includes a semi-rigid cage 220. Within the cage 220 is a balloon 222. The cage 220 may be formed of Nitinol, for example, which may expand to its configuration as illustrated once placed into the inferior vena cava. The purpose of the cage 220 is to maintain the inferior vena cava open and to anchor the balloon so that the balloon 222 may control the blood flow therethrough. The balloon is coupled to the conduit 36 which provides fluid to the balloon 222. Hence, when the venous returned blood flow is to be reduced, additional fluid is provided through the conduit 36 to the balloon 222 to increase its volume and thus reduce blood flow through the inferior vena cava 22. Conversely, when venous returned blood flow is to be increased, fluid may be removed from the balloon 222 through the conduit 36 to reduce the volume of the balloon 222. This in turn causes an increase in venous returned blood flow through the inferior vena cava.

While particular embodiments of the present invention have been shown and described, modifications may be made, and it is therefore intended in the appended claims to cover all such changes and modifications which fall within the true spirit and scope of the invention.

What is claimed:

1. A blood pressure control system comprising:
a pressure sensor that senses blood pressure of a patient; and
a blood flow regulator that varies venous return blood flow in response to the sensed blood pressure.

2. The system of claim 1 wherein the pressure sensor senses the blood pressure at spaced apart times.

3. The system of claim 1 wherein the blood flow regulator includes an inflatable balloon configured to be placed within the inferior vena cava.

4. The system of claim 1 wherein the system is totally implantable within a human body.

5. The system of claim 1 wherein the pressure sensor senses blood pressure of the left atrium.

6. The system of claim 1 wherein the blood flow regulator constricts blood flow through the inferior vena cava.

7. The system of claim 6 wherein the blood flow regulator includes an inflatable cuff configured to be placed about the inferior vena cava.

8. The system of claim 1 further comprising a processor that determines if the blood pressure sensed by the pressure sensor is above a target pressure and wherein the blood flow regulator reduces the venous return blood flow in response to the processor determining that the sensed blood pressure is above the target pressure.

9. The system of claim 8 wherein the processor computes running blood pressure averages and determines if computed running blood pressure averages are above the target pressure.

10. The system of claim 8 wherein the blood flow regulator increases the venous return blood flow if the blood pressure is below the target pressure.

11. The system of claim 8 further including an activity sensor that senses patient activity and wherein the processor determines the target pressure responsive to sensed patient activity.

12. The system of claim 8 wherein the target pressure is a fixed target pressure.

13. The system of claim 8 wherein the processor decreases the target pressure over time.

14. The system of claim 13 wherein the processor incrementally decreases the target pressure every twenty-four to seventy-two hours.

15. The system of claim 13 further including an activity sensor that senses patient activity and wherein the processor modifies the target pressure responsive to sensed patient activity.

16. The system of claim 8 wherein the processor determines mean arterial pressure responsive to the pressure sensor and at spaced apart times, computes average mean arterial pressures, and determines if computed average mean arterial pressures are above the target pressure.

17. The system of claim 16 wherein the processor averages a last predetermined number of determined mean arterial pressures to compute the average mean arterial pressures.

18. A blood pressure control system comprising:
blood pressure sensing means for sensing blood pressure of a patient; and
blood flow regulating means for varying venous return blood flow responsive to the sensed blood pressure.

19. The system of claim 18 wherein the blood pressure sensing means senses the blood pressure at spaced apart times.

20. The system of claim 18 wherein the blood flow regulating means includes an inflatable balloon configured for being placed within the inferior vena cava.

21. The system of claim 18 wherein the system is totally implantable within a human body.

22. The system of claim 18 wherein the blood pressure sensing means senses blood pressure of the left atrium.

23. The system of claim 18 wherein the blood flow regulating means includes constricting means for constricting blood flow through the inferior vena cava.

24. The system of claim 23 wherein the constricting means includes an inflatable cuff configured for being placed about the inferior vena cava.

25. The system of claim 18 further comprising comparing means for comparing the sensed blood pressure to a target blood pressure and wherein the regulating means includes means for reducing venous return blood flow when the sensed blood pressure is above the target pressure.

26. The system of claim 25 further comprising computing means for computing blood pressure running averages and wherein the comparing means compares computed blood pressure running averages to the target blood pressure.

27. The system of claim 25 wherein the blood flow regulating means increases the venous return blood flow when the sensed blood pressure is below the target blood pressure.

28. The system of claim 25 further including activity sensing means for sensing patient activity and means for determining the target blood pressure responsive to sensed patient activity.

29. The system of claim 25 wherein the target blood pressure is a fixed target pressure.

30. The system of claim 25 further comprising target pressure control means for decreasing the target pressure over time.

31. The system of claim 30 wherein the target pressure control means incrementally decreases the target pressure every twenty-four to seventy-two hours.

32. The system of claim 30 further including activity sensing means for sensing patient activity and wherein the target pressure control means modifies the target blood pressure responsive to sensed patient activity.

33. The system of claim 25 wherein the blood pressure sensing means includes means for determining mean arterial pressure responsive to the sensed blood pressure sensor and at spaced apart times, wherein the system further includes computing means for computing average mean arterial pressures, and wherein the comparing means compares average mean arterial pressures to the target pressure.

34. The system of claim 33 wherein the computing means averages a last predetermined number of determined mean arterial pressures for computing the average mean arterial pressures.

35. A method of controlling blood pressure of a patient comprising:
sensing blood pressure of the patient; and
varying venous return blood flow responsive to the sensed blood pressure.

36. The method of claim 35 wherein the step of sensing blood pressure includes sensing the blood pressure at spaced apart times.

37. The method of claim 35 wherein the reducing step includes placing an inflatable balloon within the inferior vena cava.

38. The method of claim 35 wherein each step is performed within a patient's body.

39. The method of claim 35 wherein the blood pressure sensing step includes sensing blood pressure of the left atrium.

40. The method of claim 35 wherein the reducing step includes constricting blood flow through the inferior vena cava.

41. The method of claim 40 wherein the constricting steps includes placing an inflatable cuff about the inferior vena cava.

42. The method of claim 35 including the further step of comparing the sensed blood pressure to a target blood pressure and wherein the varying step includes the step of reducing venous return blood flow when the sensed blood pressure is above the target pressure.

43. The method of claim 42 further comprising the step of computing blood pressure running averages and wherein the comparing step includes comparing computer blood pressure running averages to the target blood pressure.

44. The method of claim 42 including the further step of increasing the venous return blood flow when the sensed blood pressure is below the target blood pressure.

45. The method of claim 42 including the further steps of sensing patient activity and determining the target blood pressure responsive to sensed patent activity.

46. The method of claim 42 wherein the target blood pressure is a fixed target pressure.

47. The method of claim 42 including the further step of decreasing the target pressure over time.

48. The method of claim 47 wherein the decreasing step includes incrementally decreasing the target pressure every twenty-four to seventy-two hours.

49. The method of claim 47 including the further steps of sensing patient activity and modifying the target blood pressure responsive to sensed patient activity.

50. The method of claim 42 wherein the blood pressure sensing step includes determining, at spaced apart times, mean arterial pressure responsive to the sensed blood pressure and computing average mean arterial pressures, and wherein the comparing step includes comparing average mean arterial pressures to the target pressure.

51. The method of claim 50 wherein the computing step includes averaging a last predetermined number of determined mean arterial pressures.

52. The method of claim 40 wherein the constricting step includes the step of regulating venous return blood flow at a point distal to the renal veins.

53. A blood pressure monitor comprising a pressure sensor adapted to be mounted on a septal membrane of a heart.

54. The blood pressure monitor of claim 53 wherein the pressure sensor includes a rigid ring, a flexible diaphragm within the rigid ring, and a transducer carried by the flexible diaphragm.

55. The blood pressure monitor of claim 54 further comprising a plurality of anchors extending from the rigid ring that mount the pressure sensor to the septal membrane.

56. The blood pressure monitor of claim 53 wherein the pressure sensor provides an analog signal representing sensed blood pressure and wherein the monitor further includes an analog to digital converter that converts the analog signal to a corresponding digital signal.

57. A method of monitoring blood pressure including the steps of:
attaching a pressure sensor to a septal membrane of a heart to generate a signal representing sensed blood pressure; and
reading the signal representing the sensed blood pressure.

58. The method of claim 57 wherein the attaching step includes the step of mounting the pressure sensor to the septal membrane between the right atrium and the left ventricle.

59. The method of claim 58 wherein the pressure sensor is mounted within the right atrium.
